(19) United States
(12) Patent Application Publication
(10) Pub. No.: US 2011/0190633 A1
(43) Pub. Date: Aug. 4, 2011

(54) IMAGE PROCESSING APPARATUS, ULTRASONIC DIAGNOSTIC APPARATUS, AND IMAGE PROCESSING METHOD

(76) Inventors: Tetsuya Kawagishi, Nasushiobara (JP); Yasuhiko Abe, Otawara-shi (JP)

(21) Appl. No.: 13/018,881

(22) Filed: Feb. 1, 2011

(30) Foreign Application Priority Data
Feb. 4, 2010 (JP) 2010-023302
Dec. 27, 2010 (JP) 2010-291307

(51) Int. Cl. A61B 8/08; G06T 7/00; G06T 15/00

(52) U.S. Cl. 600/443; 382/131; 345/419

(57) ABSTRACT

According to one embodiment, an image processing apparatus includes a storage unit, setting unit, associating unit, and registering unit. The storage unit stores 2D or 3D first time series image data and second time series image data over a predetermined period. The setting unit sets a first ROI on the first image data and a second ROI on the second image data for each of a plurality of phases in the predetermined period in accordance with a user instruction or by image processing. The associating unit associates the set first ROI with the set second ROI for each of the plurality of phases. The registering unit registers the first image data and the second image data for each of the plurality of phases based on a relative positional relationship between the first ROI and the second ROI which are associated with each other.

### CROSS-REFERENCE TO RELATED APPLICATIONS

This application is based upon and claims the benefit of priority from Japanese Patent Applications No. 2010-023302, filed Feb. 4, 2010; and No. 2010-291307, filed Dec. 27, 2010; the entire contents of all of which are incorporated herein by reference.

### FIELD

Embodiments described herein relate generally to an image processing apparatus, an ultrasonic diagnostic apparatus, and an image processing method.

### BACKGROUND

Modalities capable of collecting time series volume data are coming along in recent years. Examples are 3D echography or 4D abdominal echography using an ultrasonic diagnostic apparatus and heart examination using an X-ray computed tomography apparatus. Needs are to use such different time series volume data to compare images before and after stress echo or before and after medical treatments or compare images between different modalities. However, there exists no technique of displaying moving images of the same anatomical region included in different time series volume data. This may cause a situation that images of the same region are displayed in certain phase but not in another phase. This makes it impossible to accurately compare the images of the same region.

### BRIEF DESCRIPTION OF THE DRAWINGS

FIG. 1 is a block diagram showing the arrangement of an image processing apparatus according to the embodiment;

FIG. 2 is a flowchart showing the typical procedure of image processing to be executed under the control of a control unit shown in FIG. 1;

FIG. 3 is a view for explaining setting processing, association processing, and registration processing to be performed in steps S7 to S9 of FIG. 2 concerning a remaining phase θj using interpolation;

FIG. 4 is a view for explaining setting processing, association processing, and registration processing to be performed in steps S7 to S9 of FIG. 2 concerning a remaining phase θj using automatic recognition (dictionary function);

FIG. 5 is a view for explaining setting processing, association processing, and registration processing to be performed in steps S7 to S9 of FIG. 2 concerning a remaining phase θj using tracking processing;

FIG. 6 is a view for explaining parallel display of time series wall motion image data and time series CT image data to be performed in step S11 of FIG. 2;

FIG. 7 is a view showing an example of superimposed display of time series 3D wall motion image data and time series 3D coronary artery image data to be performed on a display unit shown in FIG. 1;

FIG. 8 is a view showing an example of highlighting of a vascular region R3 running through a wall motion abnormal region to be performed on the display unit shown in FIG. 1;

FIG. 9 is a view showing an example of superimposed display of multisection time series wall motion image data and multisection time series coronary artery image data to be performed on the display unit shown in FIG. 1;

FIG. 10 is a view showing an example of superimposed display of time series 3D wall motion image data and time series X-ray contrast image data to be performed on the display unit shown in FIG. 1; and

FIG. 11 is a block diagram showing the arrangement of an ultrasonic diagnostic apparatus according to a modification of the embodiment.

### DETAILED DESCRIPTION

In general, according to one embodiment, an image processing apparatus according to the embodiment includes a storage unit, setting unit, associating unit, and registering unit. The storage unit stores 2D or 3D first time series image data and second time series image data over a predetermined period. The setting unit sets a first ROI on the first image data and a second ROI on the second image data for each of a plurality of phases in the predetermined period in accordance with a user instruction or by image processing. The second ROI is anatomically substantially the same as the first ROI. The associating unit associates the set first ROI with the set second ROI for each of the plurality of phases. The registering unit registers the first image data and the second image data for each of the plurality of phases based on a relative positional relationship between the first ROI and the second ROI which are associated with each other.

An image processing apparatus, ultrasonic diagnostic apparatus, and image processing method according to the embodiment will now be described with reference to the accompanying drawing. The image processing apparatus is a computer apparatus for assisting observation of a periodically moving examination region on a moving image. Image processing according to the embodiment is applicable to any examination region. However, to make a more specific description below, the examination region is assumed to be a heart that rhythmically repeats dilatation and contraction.

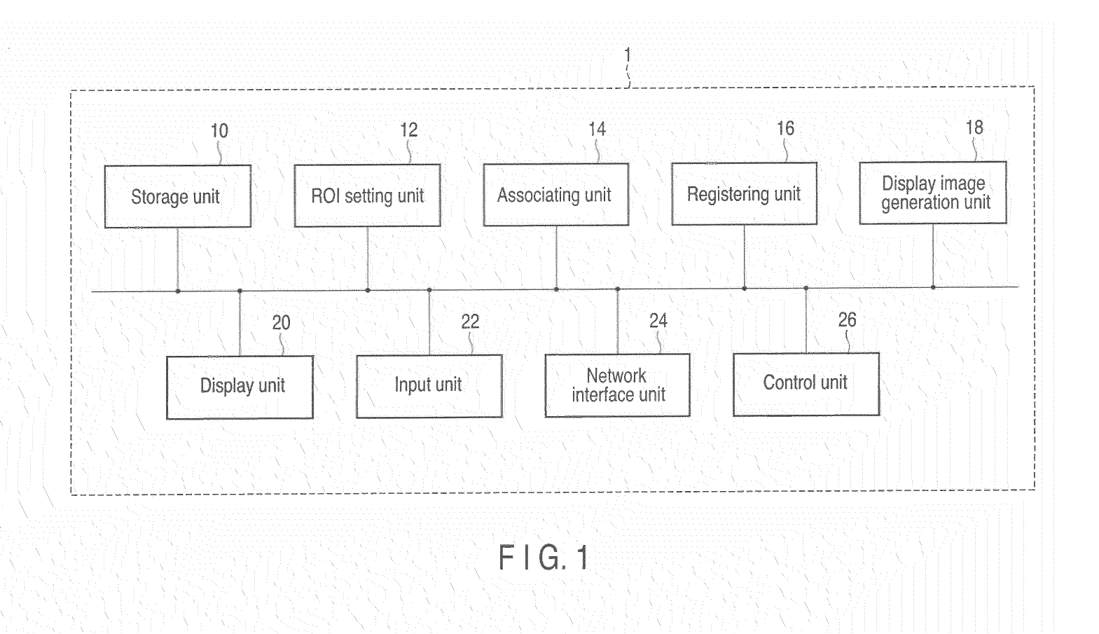

FIG. 1 is a block diagram showing the arrangement of an image processing apparatus 1 according to the embodiment. As shown in FIG. 1, the image processing apparatus 1 includes a storage unit 10, ROI setting unit 12, associating unit 14, registering unit 16, display image generation unit 18, display unit 20, input unit 22, network interface unit 24, and control unit 26.

The storage unit 10 stores at least two time series image data concerning the heart of the same object. The image data is 2D image data or 3D image data (volume data). Time series image data is a set of image data associated with a plurality of phases in a predetermined period (one or more cardiac cycles). The image data according to this embodiment may be generated by any existing medical image diagnostic apparatus such as an ultrasonic diagnostic apparatus, X-ray computed tomography apparatus (X-ray CT apparatus), X-ray diagnostic apparatus, magnetic resonance imaging apparatus, or nuclear medicine diagnostic apparatus. In the following embodiment, time series image data is assumed to be volume data for the descriptive convenience. There are two types of time series volume data. Time series volume data of one type will be referred to as first volume data, and the other type as second volume data. The storage unit 10 stores each time series volume data in association with codes representing phases. The storage unit 10 also stores an image processing program to be executed by the control unit 26.

The ROI setting unit 12 sets, for each of the plurality of phases in the predetermined period, anatomically almost the same ROI (region of interest) for the first volume data and the second volume data concerning almost the same phase. In other words, the ROI setting unit 12 sets a first ROI for the first time series volume data and a second ROI, which is anatomically almost the same as the first ROI, for the second time series volume data. Each ROI may be either manually designated by the user via the input unit 22 or specified by image processing. Note that the ROI is set by manual designation for at least one phase. For each of the remaining phases (those of the phases in the predetermined period which are not manually designated), the ROI is set by image processing.

The associating unit 14 associates the first ROI of the first volume data with the second ROI of the second volume data for each of the plurality of phases in the predetermined period in accordance with the position of the first ROI of the first volume data and that of the second ROI of the second volume data in a specific phase. The specific phase is the phase for which the ROI is set by manual designation. Association processing changes depending on whether the phase of the association processing target is the specific phase or a remaining phase (a phase for which no ROI is set by image processing). The associating unit 14 associates the first ROI of the first time series volume data with the second ROI of the second time series volume data for each phase.

The registering unit 16 registers the first volume data and the second volume data in each phase, which are associated with each other. More specifically, the registering unit 16 registers the first volume data and the second volume data for each of the plurality of phases based on the relative positional relationship between the first ROI and the second ROI which are associated with each other. The registering unit 16 thus registers the first time series volume data and the second time series volume data for each phase based on the relative positional relationship between the first ROI included in the first time series volume data and the second ROI included in the second time series volume data.

The display image generation unit 18 generates first time series display image data concerning the first ROI based on the first time series volume data after the registration. The display image generation unit 18 also generates second time series display image data concerning the second ROI based on the second time series volume data after the registration.

The display unit 20 displays the first time series display image data and the second time series display image data in parallel or in a superimposed manner on a display device as moving images. As the display device, for example, a CRT display, liquid crystal display, organic EL display, plasma display, or the like is employed.

The input unit 22 instructs to, for example, start image processing or designate the ROI in accordance with a user's input device operation instruction. As the input device, for example, a keyboard, mouse, various kinds of buttons, touch panel, or the like is employed.

The network interface unit 24 transmits/receives various kinds of image data via a medical image diagnostic apparatus or image server and a network (none are shown).

The control unit 26 functions as the main unit of the image processing apparatus 1. More specifically, the control unit 26 reads out, from the storage unit 10, the image processing program to be used to execute time series association processing of ROIs, expands it on the memory of its own, and controls the units in accordance with the expanded image processing program.

The operation of the image processing apparatus 1 according to the embodiment will be described below in detail. To make a more specific description of the operation below, the first volume data is assumed to be volume data concerning wall motion information (to be referred to as wall motion volume data hereinafter) generated by an ultrasonic diagnostic apparatus. As is known, the wall motion volume data is generated in the following way. First, the ultrasonic diagnostic apparatus repetitively three-dimensionally scans the heart of the object by ultrasonic waves via an ultrasonic probe, thereby generating time series ultrasonic volume data. Next, the ultrasonic diagnostic apparatus extracts the myocardial region from the generated time series ultrasonic volume data by 3D speckle tracking. The ultrasonic diagnostic apparatus then analyzes the wall motion in the extracted myocardial region to calculate wall motion information. The ultrasonic diagnostic apparatus assigns the calculated wall motion information to a voxel to generate wall motion volume data. Note that the wall motion information represents parameters such as displacement, displacement ratio, distortion, distortion ratio, moving distance, velocity, and velocity gradient for a predetermined direction of cardiac muscle. The wall motion volume data represents the spatial distribution of these pieces of wall motion information.

The second volume data is assumed to be volume data concerning the coronary arteries (to be referred to as CT volume data hereinafter) generated by an X-ray CT apparatus. The X-ray CT apparatus repetitively scans the coronary arteries with an injected contrast medium by X-rays, thereby generating time series CT volume data.

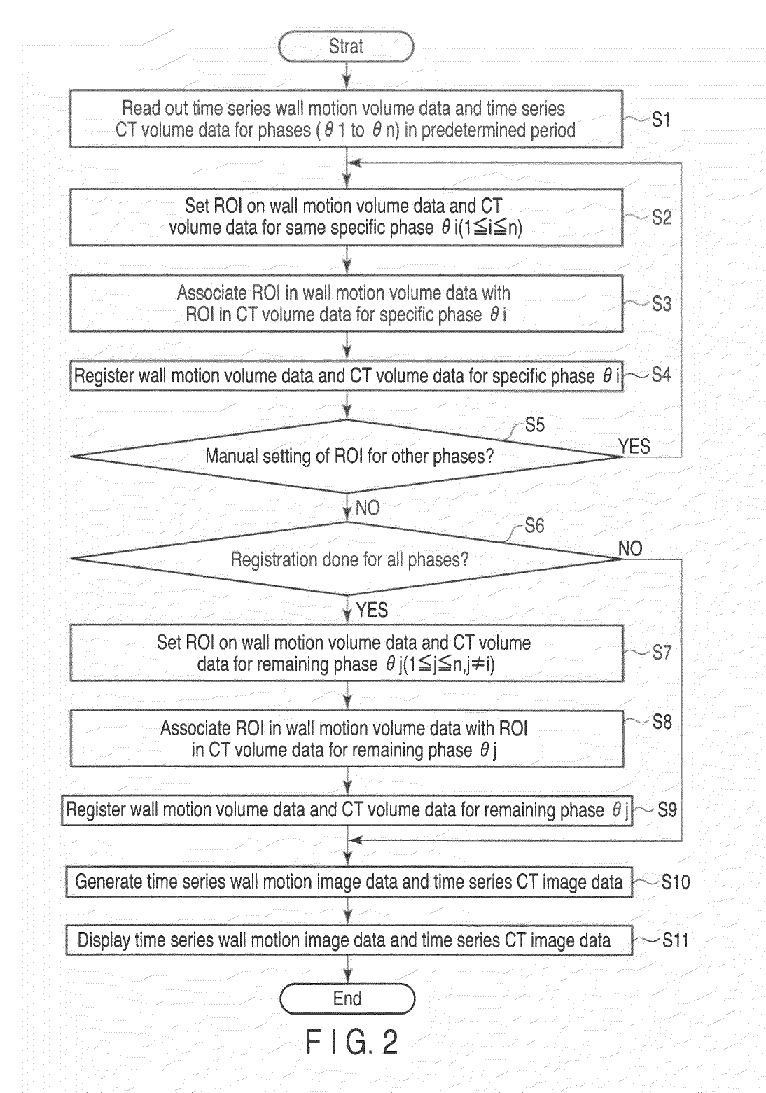

For the descriptive convenience, the initial phase will be referred to as “θ1”, and the terminal phase as “θn” (n is an integer of 2 or more), where n indicates the nth phase counted from the initial phase θ1. In general, the time resolution of the time series wall motion volume data is different from that of the time series CT volume data. Typically, the time resolution of the time series wall motion volume data is higher than that of the time series CT volume data. In this embodiment, however, the two volume data are assumed to have the same time resolution for the descriptive convenience. FIG. 2 is a flowchart showing the typical procedure of image processing to be executed under the control of the control unit 26. As shown in FIG. 2, upon receiving a user's image processing start instruction via the input unit 22, the control unit 26 reads out time series wall motion volume data and time series CT volume data in a predetermined period from the storage unit 10 (step S1). The control unit 26 supplies the readout time series wall motion volume data and time series CT volume data to the ROI setting unit 12.

When step S1 is done, the control unit 26 causes the ROI setting unit 12 to perform ROI setting processing (step S2). In step S2, the region-of-interest setting unit ROI sets ROIs that are anatomically almost the same for the wall motion volume data and CT volume data concerning almost the same specific phase θi (1≦I≦n). A ROI set for the wall motion volume data will be referred to as a wall motion ROI, and a ROI set for the CT volume data, as a CT ROI.

The setting processing in step S2 will be described below in detail. In step S2, typically, ROIs are set by user's manual designation via the input unit 22. Manual designation is performed on the display image displayed on the display unit 20. For example, a plurality of feature points are designated on a wall motion display image based on the wall motion volume data. The section of the wall motion display image is set on an arbitrary section of the wall motion volume data. The feature points are designated as, for example, a plurality of points such as three points that are not arranged on a line. For example, to observe a myocardial motion abnormality, the feature points are designated in the myocardial motion abnormal region. When the user has designated the plurality of feature points, the ROI setting unit 12 sets a wall motion ROI on the region including the plurality of designated feature points. For example, the ROI setting unit 12 sets a wall motion ROI on the region surrounded by the designated feature points. When a region of clinical interest on an image is found in a focal region such as an ischemia region, a lesion region and the like, the designated feature points are set to surround a relatively narrow region. When a region of clinical interest is a relatively broad in an image, the designated feature points are set to surround a relatively broad region to cover the region of clinical interest. In this case, a ROI is set on the relatively broad region in the image. A designated feature point may be set as the wall motion ROI. Next, the user designates, via the input unit 22 on a CT display image based on the CT volume data, a plurality of corresponding points corresponding to the plurality of feature points set in the wall motion volume data. The section of the CT display image is set on an arbitrary section in the CT volume data. When the user has designated the plurality of corresponding points, the ROI setting unit 12 sets the plurality of designated corresponding points as ROIs of CT. The positions of the set wall motion ROIs and CT ROIs are supplied to the associating unit 14 in association with the specific phase θi.

The specific phase θi can arbitrarily be designated by the user via the input unit 22. For example, if electrocardiographic data is associated with the time series wall motion volume data and the time series CT volume data, the specific phase θi is designated using the electrocardiogram. For example, the user designates, via the input unit 22, the phase θi on the electrocardiogram displayed on the display unit 20. After the phase θi is designated, the control unit 26 sets the designated phase θi as the specific phase θi. Thus designating the specific phase θi in synchronism with the electrocardiogram allows to more accurately detect the same phase. If no electrocardiographic data is associated, the user may designate the specific phase θi via the input unit 22 by visually confirming, for example, the open/close timing of the cardiac valve or the endosystolic and endodiastolic timings or the like.

When step S2 is done, the control unit 26 causes the associating unit 14 to perform association processing (step S3). In step S3, the associating unit 14 associates the ROIs set for the wall motion volume data and the CT volume data concerning the specific phase θi with each other. The positions of the wall motion ROIs and those of the CT ROIs which are associated with each other are stored in the storage unit 10 in association with each other.

When step S3 is done, the control unit 26 causes the registering unit 16 to perform registration processing (step S4). In step S4, the registering unit 16 calculates registration information concerning the specific phase θi based on the relative positional relationship between the wall motion ROIs of and the CT ROIs which are associated with each other. The registering unit 16 registers the wall motion volume data and the CT volume data concerning the specific phase θi in accordance with the calculated registration information. The registration information represents, for example, the relative position, relative direction, and relative scale between the wall motion ROIs and the CT ROIs. In other words, the registration information represents vectors that connect the wall motion ROIs to the CT ROIs. More specifically, the registration information represents the coordinate transformation from the wall motion volume data to the CT volume data or the coordinate transformation from the CT volume data to the wall motion volume data. The registering unit 16 multiplies the wall motion volume data or the CT volume data by the calculated coordinate transformation, thereby registering the wall motion volume data and the CT volume data.

When step S4 is done, the control unit 26 waits for an instruction about whether or not to set ROIs for other phases as well by manual designation (step S5). When the user has input, via the input unit 22, an instruction to designate ROIs for other phases as well (YES in step S5), the process returns to step S2. In this way, the wall motion ROIs and the CT ROIs concerning a plurality of specific phases θi that are different from each other are repetitively associated with each other and registered. The plurality of specific phases θi may be either discrete or continuous in terms of time.

When the user has input, via the input unit 22, an instruction not to designate ROIs for other phases as well in step S5 (NO in step S5), the control unit 26 determines whether registration has been done for all phases θ1 to θn (step S6). Upon determining that registration has been done for all phases θ1 to θn (YES in step S6), the control unit 26 advances to step S10.

On the other hand, if it is determined in step S6 that a phase (remaining phase θj (1≦j≦n, j≠i)) for which registration has not been done remains (NO in step S6), the control unit 26 causes the ROI setting unit 12 to perform setting processing of ROIs for the remaining phase θj (step S7). In step S7, the ROI setting unit 12 sets wall motion ROIs on the wall motion volume data concerning the remaining phase θj based on the positions and shapes of the wall motion ROIs of the wall motion volume data concerning the specific phase θi set in step S2. In a similar manner, the ROI setting unit 12 sets CT ROIs on the CT volume data concerning the remaining phase θj based on the positions and shapes of the CT ROIs of the CT volume data concerning the specific phase θi.

When step S7 is done, the control unit 26 causes the associating unit 14 to perform association processing of ROIs for the remaining phase θj (step S8). In step S8, the associating unit 14 associates the wall motion ROIs with the CT ROIs concerning the remaining phase θj based on the relative positional relationship between the wall motion ROIs and the CT ROIs set in step S7.

When step S8 is done, the control unit 26 causes the registering unit 16 to perform registration processing of ROIs for the remaining phase θj (step S9). In step S9, the registering unit 16 registers the wall motion volume data and the CT volume data concerning the phase θj based on the relative positional relationship between the wall motion ROIs and the CT ROIs associated in step S8. The processing in steps S7, S8, and S9 will be described below in detail.

The setting processing in step S7, the association processing in step S8, and the registration processing in step S9 can adopt various methods which are roughly classified into three. The three methods will be explained below.

### Method 1: (Interpolation or Extrapolation)

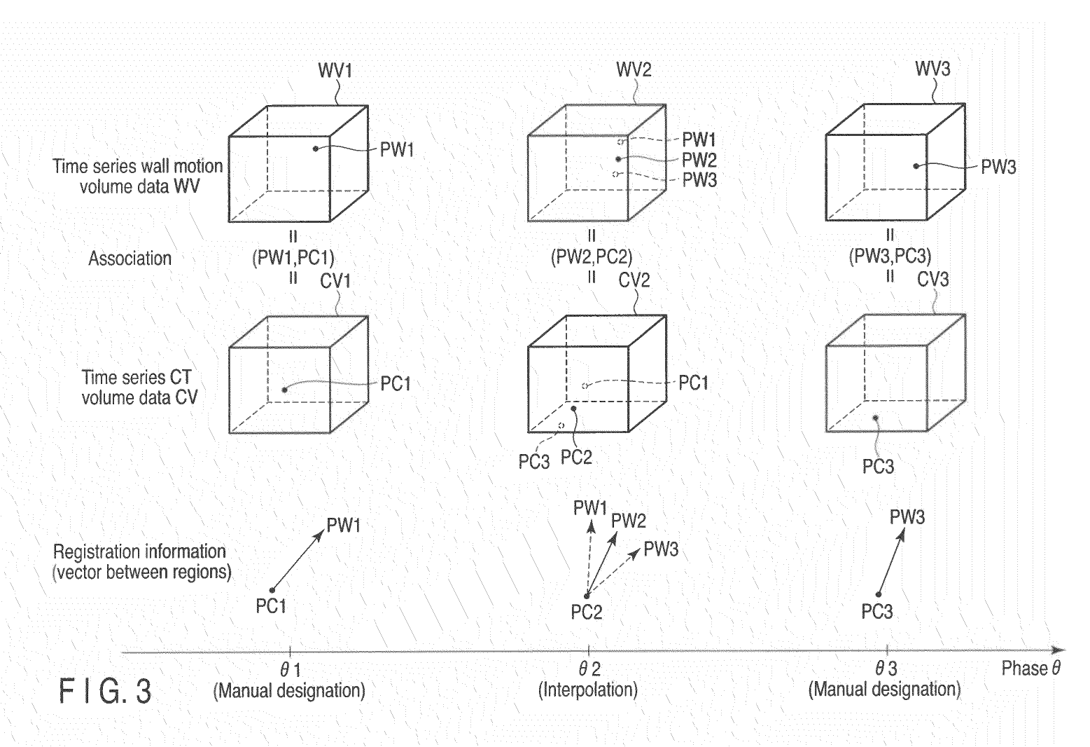

FIG. 3 is a view for explaining setting processing, association processing, and registration processing concerning the remaining phase θj using interpolation. As shown in FIG. 3, time series wall motion volume data WV and time series CT volume data CV for phases θ1, θ2, and θ3 will specifically be exemplified. Note that the temporal course of the phases θ1, θ2, and θ3 is θ1→θ2→θ3.

Assume that in step S2, a region PW1 of interest of wall motion is set in wall motion volume data WV1 concerning the phase θ1 by manual designation, whereas a region PC1 of interest of CT is set in CT volume data CV1 concerning the phase θ1 by manual designation. In step S3, the associating unit 14 associates the regions PW1 and PC1 for the phase θ1 with each other. The associated regions (PW1 and PC1) are stored in the storage unit 10 in association with each other. In step S4, the registering unit 16 calculates the registration information of the regions PW1 and PC1 (for example, the vector (relative position and direction) from the region PC1 to the region PW1) based on the relative positional relationship between them. For the phase θ3 as well, regions PW3 and PC3 are set by manual designation in step S2, and the associating unit 14 associates the regions PW3 and PC3 with each other in step S3. In step S4, the registering unit 16 calculates the registration information of the regions PW3 and PC3 (the vector from the region PC3 to the region PW3).

For the phase θ2 (remaining phase), ROIs are set by interpolation in step S7. First, the candidate position of a wall motion ROI PW2 in wall motion volume data WV2 for the phase θ2 is calculated by interpolation based on the position of the wall motion ROI PW1, the position of the wall motion ROI PW3, and the elapsed time from the phase θ1 to the phase θ3. The interpolation method may be linear interpolation or higher-order interpolation represented by spline interpolation and Lagrange interpolation. The ROI setting unit 12 sets the wall motion ROI PW2 at the calculated candidate position. The ROI setting unit 12 similarly sets a CT ROI PC2 in CT volume data CV2 for the phase θ2. Note that the method of calculating the candidate position of a ROI is not limited to interpolation. For example, the positions of ROIs for the remaining phase may be calculated by extrapolation based on the positions of the ROIs for the specific phase θ1 and the elapsed time from the phase θ1 to the phase θ2.

In step S8, the associating unit 14 associates the wall motion ROI PW2 with the CT ROI PC2. The associated regions (PW2 and PC2) are stored in the storage unit 10. In step S9, the registering unit 16 calculates the coordinate transformation from the ROI PW3 to the ROI PC3 based on the vector between the ROI PW2 and ROI PC2. The registering unit 16 multiplies the wall motion volume data WV2 by the calculated coordinate transformation, thereby registering the wall motion volume data WV2 and the CT volume data CV2. Step S9 thus ends.

The ROIs for the phase θ2 are set, associated, and registered by interpolation in the above-described way. The same processing is performed for the remaining phases. The wall motion volume data and CT volume data are thus registered for all phases θj other than the specific phase θi in the predetermined period.

Note that in the above description, the time series wall motion volume data and the time series CT volume data have the same time resolution. However, the embodiment is not limited to this. In case of different time resolutions, the ROI setting unit 12 calculates the positions of ROIs for the wanted phase by interpolation or extrapolation.

### Method 2: Automatic Recognition (Dictionary Function)

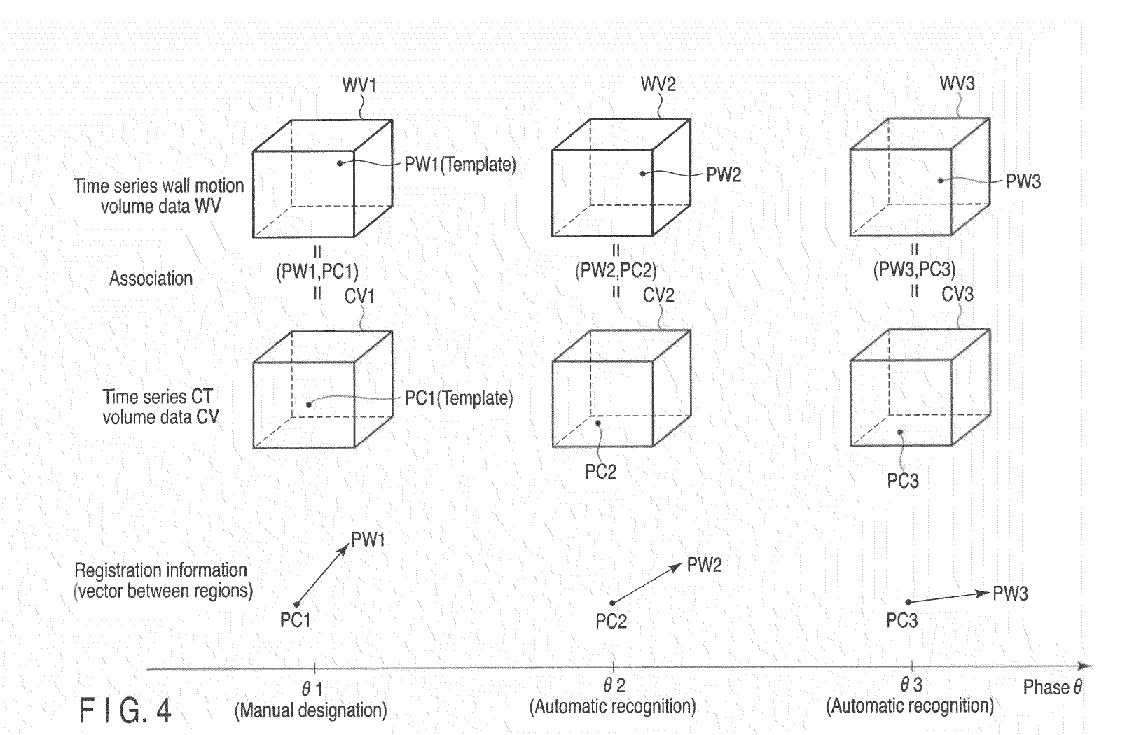

FIG. 4 is a view for explaining setting processing, association processing, and registration processing concerning the remaining phase θj using automatic recognition. Note that the same reference symbols as in FIG. 3 have the same meanings in FIG. 4. In FIG. 4, however, assume that manual designation of ROIs is performed for only the phase θ1.

This method specifies the ROIs for the remaining phases θ2 and θ3 by automatic recognition, and setting processing, association processing, and registration processing are executed in accordance with the specified ROIs. In this case, ROIs are set for each of the wall motion volume data WV and the CT volume data CV for each phase.

More specifically, in step S7, the ROI setting unit 12 performs template matching processing of the wall motion volume data WV2 using the pixel value distribution of the region PW1 of interest of wall motion concerning the phase θ1 as a template, thereby specifying the region PW2 of interest of wall motion concerning the phase θ2 by automatic recognition. Similarly, the ROI setting unit 12 performs template matching processing of the CT volume data CV2 using the pixel value distribution of the region PC1 of interest of CT concerning the phase θ1 as a template, thereby specifying the region PC2 of interest of CT concerning the phase θ2 by automatic recognition. For example, assume that a ROI is set on the annulus of heart valve in each of the wall motion volume data WV1 and the CT volume data CV1 for the phase θ1. In this case, the annulus of heart valve is specified in each of the wall motion volume data WV2 and the CT volume data CV2 for the phase θ2. The ROI setting unit 12 sets the wall motion ROI PW2 of on the annulus of heart valve in the wall motion volume data WV2. The ROI setting unit 12 sets the CT ROI PC2 on the annulus of heart valve in the CT volume data CV2.

In step S8, the associating unit 14 associates the region PW2 of interest of wall motion with the region PC2 of interest of CT. In step S9, the registering unit 16 registers the wall motion volume data WV2 and the CT volume data CV2 based on the positional relationship between the regions PW2 and PC2 (the vector from the region PC2 to the region PW2).

When the ROIs for the phase θ2 are thus set, associated, and registered using automatic recognition, the same processing is performed for the next phase θ3. This processing is repeated for all phases θj other than the specific phase θi in the predetermined period. The wall motion volume data and CT volume data are thus registered for all phases θj.

### Method 3: (Tracking Processing)

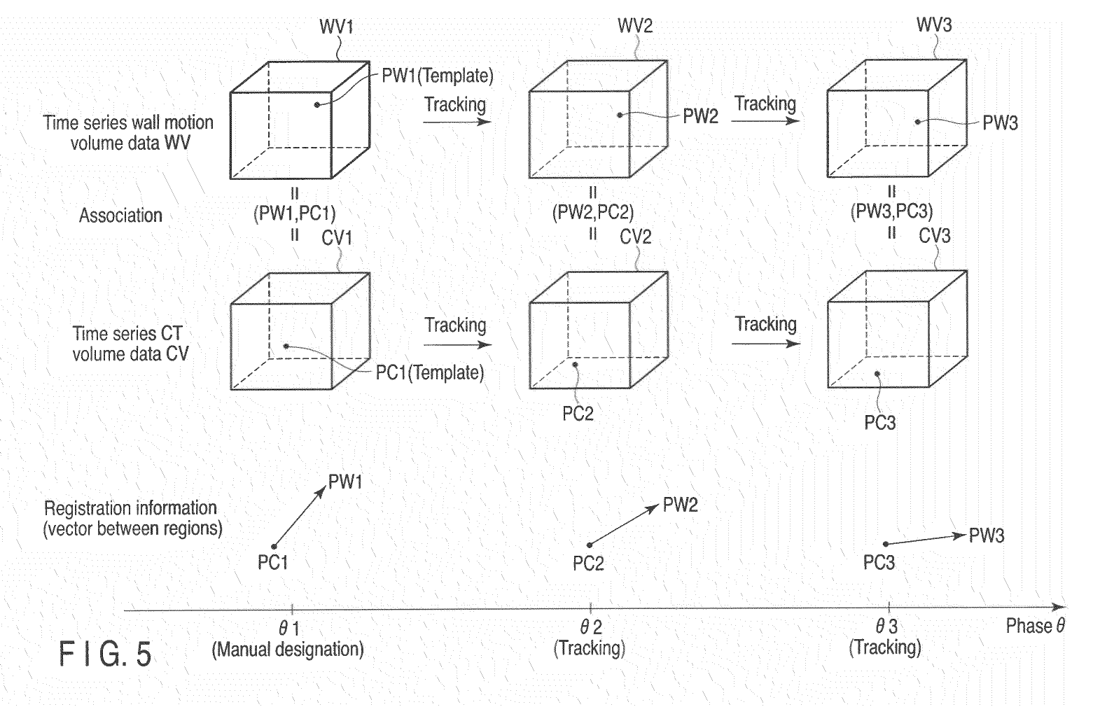

FIG. 5 is a view for explaining setting processing, association processing, and registration processing concerning the remaining phase θj using tracking processing. Note that the same reference symbols as in FIG. 3 have the same meanings in FIG. 5. In FIG. 5, however, assume that manual designation of ROIs is performed for only the phase θ1.

This method tracks ROIs set for the specific phase θ1 throughout time series volume data, and performs setting processing, association processing, and registration processing in accordance with the tracked ROIs. In this case, association processing and registration processing are executed after the ROIs have been set for the remaining phases θ2 and θ3.

More specifically, in step S7, the ROI setting unit 12 performs template matching processing of the wall motion volume data WV2 and WV3 using the pixel value distribution of the wall motion ROI PW1 concerning the phase θ1 as a template, thereby specifying the wall motion ROI PW2 and ROI PW3 by tracking. Similarly, the ROI setting unit 12 performs template matching processing of the CT volume data CV2 and CV3 using the pixel value distribution of the CT ROI PC1 concerning the phase θ1 as a template, thereby specifying the CT ROI PC2 and PC3 by tracking. The ROI setting unit 12 sets the specified wall motion ROIs and CT ROIs.

In step S8, the associating unit 14 associates the wall motion PW2 with the CT ROI PC2. Similarly, the associating unit 14 associates the wall motion PW3 with the CT ROI PC3. In step S9, the registering unit 16 registers the wall motion volume data WV2 and the CT volume data CV2 based on the vector from the CT ROI PC2 to the wall motion PW2. Similarly, the registering unit 16 registers the wall motion volume data WV3 and the CT volume data CV3 based on the vector from the CT ROI PC3 to the wall motion PW3.

In the above-described way, the ROIs are set for all the remaining phases θj in the predetermined period using tracking processing. Then, the ROIs for each of the remaining phases θj are associated with each other, and the wall motion volume data and the CT volume data for each of the remaining phases θj are registered.

The processing in steps S7, S8, and S9 has been described above. At the timing of the end of step S9, the time series wall motion volume data and the time series CT volume data are registered for each phase.

Generally, for determining parallel translation, rotation and expansion-and-contraction of a 3D image, registration is performed using three or more points. For this registration, the least squares method and the like are employed.

When it is determined in step S6 that registration has been done for all phases θ1 to θn, or when step S9 is performed, the control unit 26 causes the display image generation unit 18 to execute image generation processing (step S10). In step S10, the display image generation unit 18 performs 3D image processing of the registered time series wall motion volume data, thereby generating time series wall motion image data. Similarly, the display image generation unit 18 performs 3D image processing of the registered time series CT volume data, thereby generating time series CT image data. The generated time series wall motion image data and time series CT image data have been registered. Examples of the 3D image processing are MPR (Multi Planar Reconstruction) processing, volume rendering, surface rendering, MIP (Maximum Intensity Projection), CPR (Curved Planar Reconstruction) processing, and SPR (Stretched CPR) processing.

When step S10 is done, the control unit 26 causes the display unit 20 to perform display processing (step S11). In step S11, the display unit 20 displays the generated time series wall motion image data and time series CT image data as dynamic images. The display methods are roughly classified into parallel display and superimposed display. In the case of superimposed display, the display unit 20 displays time series wall motion image data and the time series CT image data while superimposing time series wall motion image data on the time series CT image data.

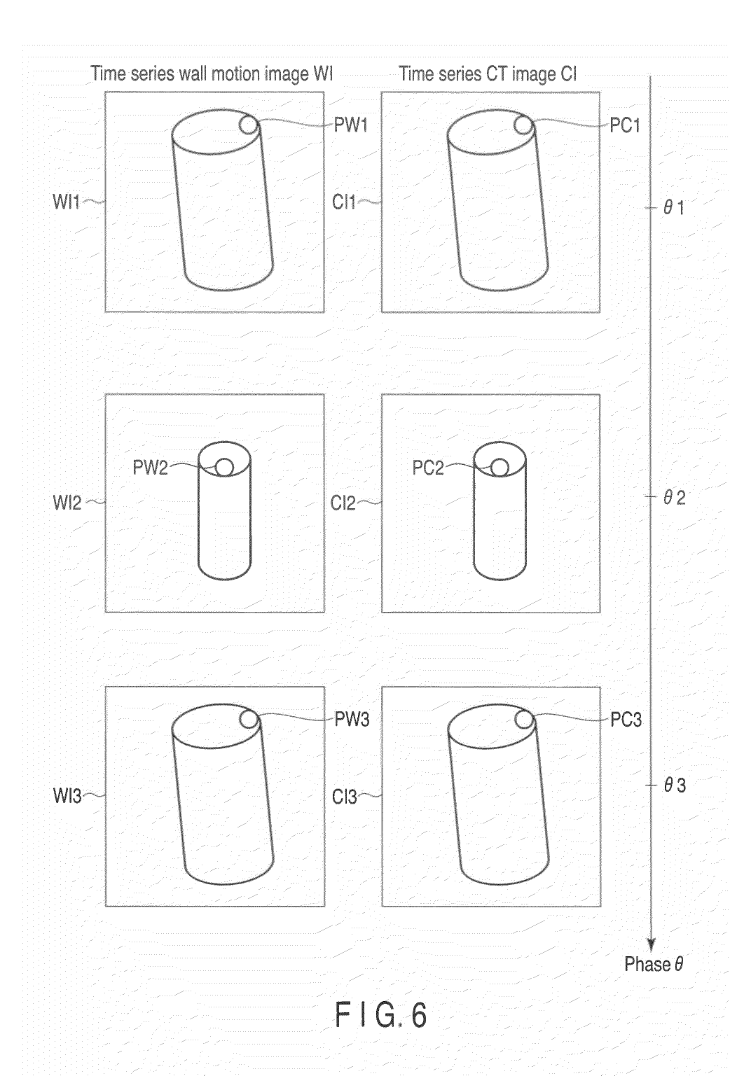

FIG. 6 is a view for explaining parallel display of time series wall motion image data WI and time series CT image data CI. As shown in FIG. 6, the region PW of interest of wall motion of the wall motion image data WI and the region PC of interest of CT of the CT image data CI are registered for each phase θ. Hence, the region PW of interest of wall motion and the region PC of interest of CT can be displayed at the same position on the images for all phases θ. This avoids the conventional situation that images of the same region are displayed in certain phase but not in another phase.

The heart that is the examination region of this embodiment vigorously moves in the body while repeating contraction and dilatation. In particular, when scanning the heart by the ultrasonic diagnostic apparatus, the operator scans while moving the ultrasonic probe. In this case, the position of the ROI in the cardiac region included in the volume data largely changes on the image for each phase.

The image processing apparatus 1 associates the ROI in the time series wall motion volume data with that in the CT volume data for each phase. The image processing apparatus 1 calculates the registration information between the wall motion volume data and the CT volume data for each phase, and registers the wall motion volume data and the CT volume data for each phase based on the calculated registration information. The image processing apparatus 1 can accurately register the ROIs and display them as moving images by registering for each phase even when the examination region vigorously moves. For this reason, the user can easily do comparison interpretation to, for example, confirm, on a CT moving image, an abnormal region in the wall motion moving image. That is, the user can accurately assess the wall motion of the ROI by observing the ROIs time-serially registered between the wall motion volume data and the CT volume data.

Note that the size of the ROI on the time series wall motion image data and that of the ROI on the time series CT image data may sometimes be different. In this case, the display unit 20 changes the size of the wall motion image data or that of the CT image data for each phase based on the relative positional relationship between the two ROIs so as to equalize their sizes. More specifically, the pixel size of the wall motion image data or the CT image data is enlarged or reduced.

To improve the convenience of observation, the display unit 20 can fix the display section of one of the display image data and make that of the other follow the fixed section. More specifically, the display unit 20 first fixes the position of the display section of the time series wall motion image data. Next, the display unit 20 calculates the position of the display section of the time series CT image data, which is anatomically almost the same as the fixed display section, for each phase based on the time series registration information. The display unit 20 then generates time series CT image data from the time series CT volume data in accordance with the display section position calculated for each phase. The display unit 20 displays the generated time series CT image data and time series wall motion image data as moving images. This enables the display section of the time series CT image data to follow the fixed section of the time series wall motion image data.

Three display examples of first time series display image data and second time series display image data according to this embodiment will be described next. Superimposed display of time series 3D wall motion image data and time series 3D coronary artery image data will be explained as the first display example. The 3D wall motion image data is functional image data generated by volume-rendering wall motion volume data. The 3D wall motion image data includes a wall motion abnormal region. The abnormal region is a set of pixels each having wall motion information larger or smaller than a preset threshold. The 3D coronary artery image data is structural image data generated by volume-rendering CT volume data. The 3D coronary artery image data includes a cardiac region. The cardiac region includes a coronary artery region.

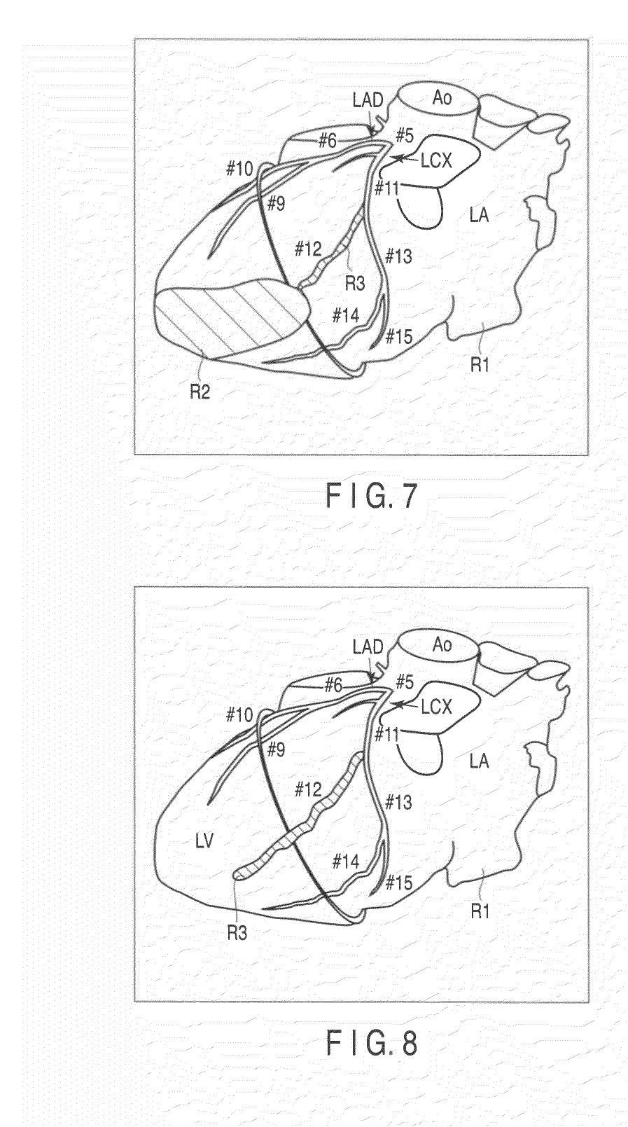

FIG. 7 is a view showing an example of superimposed display of time series 3D wall motion image data and time series 3D coronary artery image data by the display unit 20. As shown in FIG. 7, a wall motion abnormal region R2 derived from the 3D wall motion image data is aligned and superimposed on a cardiac region R1 derived from the 3D coronary artery image data. This allows the user to confirm the whereabouts of the wall motion abnormality on the moving image.

Angiostenosis is known as a cause of a wall motion abnormality. Hence, the display unit 20 can highlight the vascular region running through the wall motion abnormal region R2 for the clinical convenience. The highlighted vascular region is derived from CT volume data. For example, a vascular region R3 labeled “#12” in FIG. 7 runs through the wall motion abnormal region R2 in terms of anatomical positional relationship. In this case, the vascular region R3 includes an angiostenosis region at high probability. To confirm whether the vascular region includes an angiostenosis region is clinically very important.

FIG. 8 is a view showing an example of highlighting of the vascular region R3 running through the wall motion abnormal region. As shown in FIG. 8, the display unit 20 changes the display method of the vascular region R3 derived from the 3D coronary artery image data in order to highlight it. The display unit 20 can display the vascular region R3 in a color different from that of other vascular regions to highlight it. Note that the highlighting technique is not limited to this. For example, the display unit 20 may change the lightness and saturation of the vascular region R3 or flash it. Thus highlighting the vascular region R3 running through the wall motion abnormal region R2 allows the user to easily identify the blood vessel that leads to the wall motion abnormality. In addition, the user can readily confirm matching between the wall motion abnormality and coronary stenosis. For this reason, superimposed display of the time series 3D coronary artery image data and the time series 3D wall motion image data greatly helps ischemia diagnosis. Note that the highlighting can also be done simultaneously with the superimposed display in FIG. 7.

Superimposed display of multisection time series wall motion image data and multisection time series coronary artery image data will be explained next as the second display example. The wall motion image data is functional image data generated by MPR-processing wall motion volume data. The wall motion image data includes a wall motion abnormal region. The coronary artery image data is structural image data generated by MPR-processing CT volume data. The coronary artery image data includes a cardiac region and a coronary artery region.

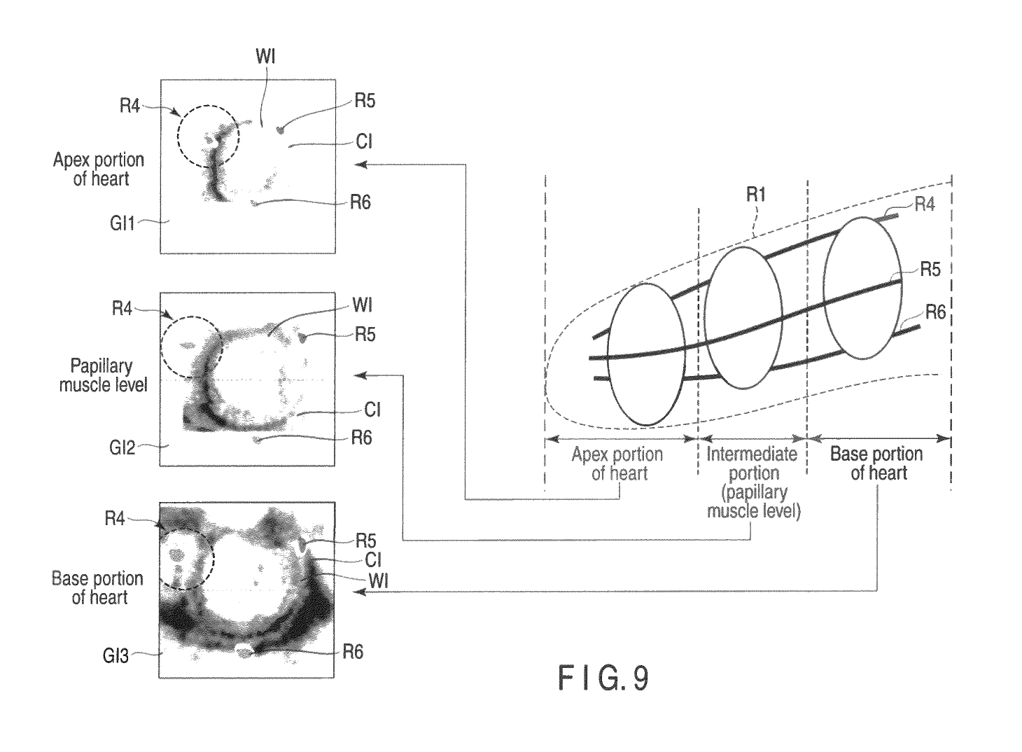

FIG. 9 is a view showing an example of superimposed display of multisection time series wall motion image data and multisection time series coronary artery image data. As shown in FIG. 9, the section positions of each image data are set at the apex portion, intermediate portion (papillary muscle level), and base portion of the heart in the cardiac region R1. Note that the cardiac region R1 is extracted from the CT volume data. The cardiac region R1 includes coronary artery regions R4, R5, and R6. As shown in FIG. 9, the display unit 20 displays superimposed image data GI1 of wall motion image data and coronary artery image data for the apex portion of the heart, superimposed image data GI2 of wall motion image data and coronary artery image data for the intermediate portion, and superimposed image data GI3 of wall motion image data and coronary artery image data for the base portion of the heart as moving images beside each other. Note that the user can change the section positions of each image data via the input unit 22.

Each superimposed image data GI includes part of of the coronary artery regions R4, R5, and R6. Of the coronary artery regions R4, R5, and R6, a coronary artery region determined by the X-ray CT apparatus to be suspected of including coronary stenosis is highlighted in a different color, lightness, saturation, or the like. For example, assume that it is determined that the coronary artery region R4 is suspected of including stenosis, as shown in FIG. 9. In this case, the display unit 20 displays the coronary artery region R4 in, for example, a color different from that of the remaining coronary artery regions R5 and R6. As another example of highlighting, the peripheral region of the coronary artery region R4 to be highlighted may be highlighted. The user can arbitrarily set the range of the peripheral region via the input unit 22.

The display unit 20 may also highlight a coronary artery region included in the wall motion abnormal region. For example, the user may designate (click) a coronary artery region on the superimposed image data via the input unit 22 so that the designated coronary artery region is highlighted in a different color or the like. Thus highlighting the vascular region running through the wall motion abnormal region allows the user to readily confirm matching between the wall motion abnormality and coronary stenosis.

Superimposed display of time series 3D wall motion image data and time series X-ray contrast image data will be explained next as the third display example. The 3D wall motion image data is functional image data generated by volume-rendering wall motion volume data. The X-ray contrast image data is structural image data generated by imaging an object with an injected contrast medium by X-rays.

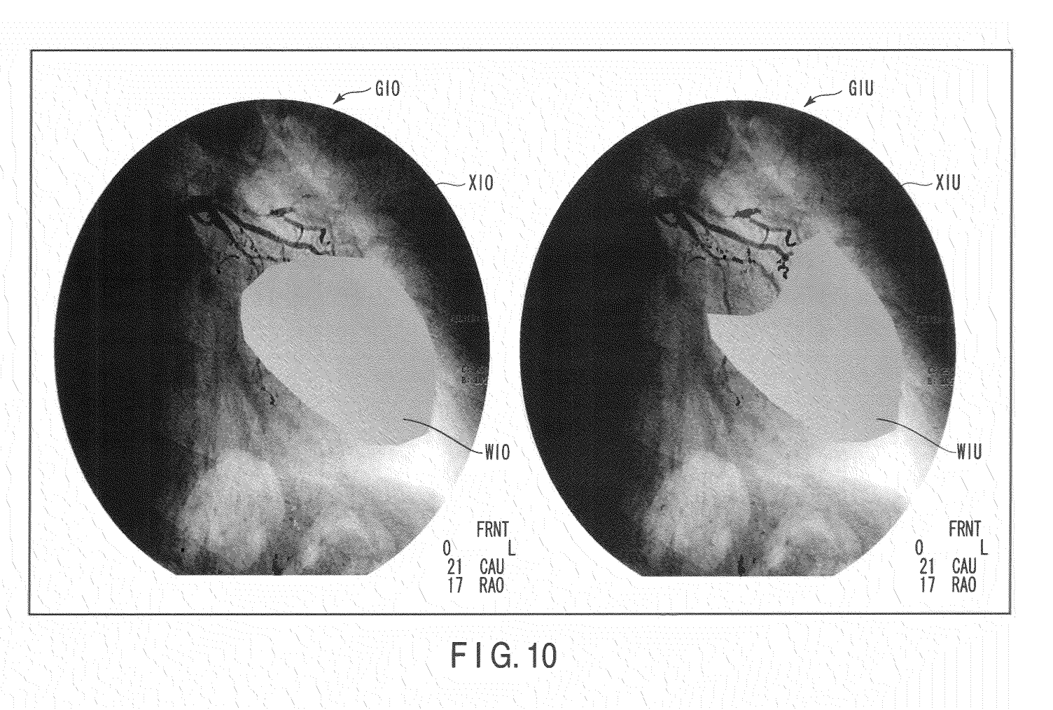

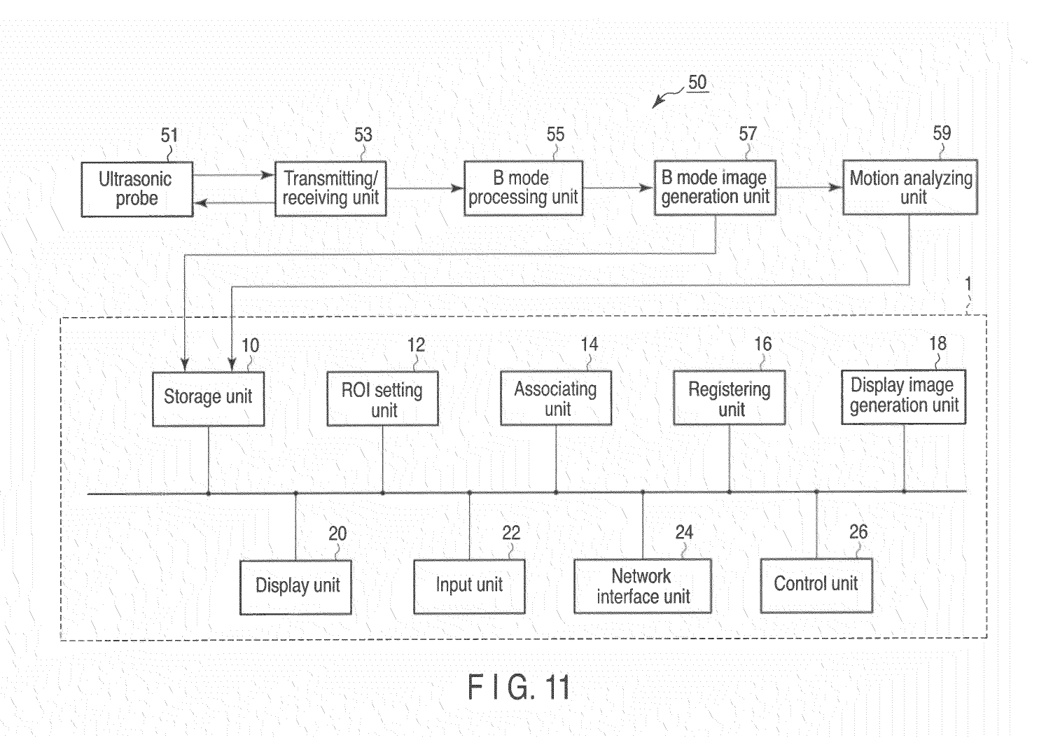

FIG. 10 is a view showing an example of superimposed display of time series 3D wall motion image data and time series X-ray contrast image data. As shown in FIG. 10, the display unit 20 displays superimposed image data GIO for the front side of the heart and superimposed image data GIU for the rear side of the heart in parallel. X-ray contrast image data XIO of the superimposed image data GIO is identical to X-ray contrast image data XIU of the superimposed image data GIU. 3D wall motion image data WIO of the superimposed image data GIO is generated by volume-rendering the wall motion volume data at a viewpoint set outside the cardiac region. 3D wall motion image data WIU of the superimposed image data GIU is generated by volume-rendering the wall motion volume data at a viewpoint set inside the cardiac region. Thus parallelly displaying the superimposed image data on the front side of the heart and that on the rear side as moving images allows the user to clearly grasp which side of the heart has the abnormality, the front side or the rear side.

As still another display example, volume data collected by the ultrasonic diagnostic apparatus before stress echo may be set as first volume data, and volume data collected by the ultrasonic diagnostic apparatus after stress echo may be set as second volume data. A declaration will be made here that the first volume data and the second volume data in this case are involved in the same examination region.

As described above, according to the embodiment, it is possible to provide an image processing apparatus and method capable of easily comparing identical portions included in different time series image data.

### (Modification)

An ultrasonic diagnostic apparatus may be equipped with the image processing apparatus 1 according to the embodiment. The ultrasonic diagnostic apparatus will be described below. Note that the same reference numerals as in the embodiment denote constituent elements having almost the same functions in the following description, and a repetitive description will be made only when needed.

Referring to FIG. 11, an ultrasonic diagnostic apparatus 50 according to the modification comprises an ultrasonic probe 51, transmitting/receiving unit 53, B-mode processing unit 55, B-mode image generation unit 57, motion analyzing unit 59, and image processing apparatus 1.

The ultrasonic probe 51 receives a driving signal from the transmitting/receiving unit 53 and transmits ultrasonic waves to the examination region (heart) of the object. The transmitted ultrasonic waves are focused into a beam. The transmitted ultrasonic waves are reflected by the examination region of the object. The reflected ultrasonic waves are received by the ultrasonic probe. The ultrasonic probe 51 generates an electrical signal (echo signal) corresponding to the strength of the received ultrasonic waves. The ultrasonic probe 51 is connected to the transmitting/receiving unit 53 via a cable. The echo signal is supplied to the transmitting/receiving unit 53.

The transmitting/receiving unit 53 repetitively scans the examination region of the subject by ultrasonic waves via the ultrasonic probe 51. More specifically, the transmitting/receiving unit 53 supplies the driving signal to the ultrasonic probe 51 to make it transmit beam-shaped ultrasonic waves. The transmitting/receiving unit 53 delays the echo signal from the ultrasonic probe 51 and adds the delayed echo signals. An electrical signal (reception signal) that forms a reception beam is formed by the delay processing and the addition processing. The reception signal is supplied to the B-mode processing unit 55.

The B-mode processing unit 55 performs B-mode processing for the reception signal. More specifically, the B-mode processing unit 55 performs logarithmic compression or envelope detection processing of the reception signal. The reception signal that has undergone the logarithmic compression or envelope detection processing is called a B-mode signal. The B-mode signal is supplied to the B-mode image generation unit 57.

The B-mode image generation unit 57 generates 2D or 3D time series B-mode image data concerning the subject based on the B-mode signal. The time series B-mode image data is supplied to the storage unit 10 and the motion analyzing unit 59. For a more detailed description, the B-mode image data is assumed to be 3D image data, that is, B-mode volume data.

The motion analyzing unit 59 performs motion analysis of the time series B-mode volume data to generate time series wall motion volume data. More specifically, the motion analyzing unit 59 extracts the myocardial region from the time series B-mode volume data by 3D speckle tracking. The motion analyzing unit 59 then analyzes the wall motion in the extracted myocardial region to calculate wall motion information. The motion analyzing unit 59 assigns the calculated wall motion information to a voxel to generate wall motion volume data. Note that the wall motion information represents parameters such as displacement, displacement ratio, distortion, distortion ratio, moving distance, velocity, and velocity gradient for a predetermined direction of cardiac muscle. The wall motion volume data is supplied to the storage unit 10.

The image processing apparatus 1 included in the ultrasonic diagnostic apparatus 50 has the same arrangement as the image processing apparatus 1 according to the embodiment. More specifically, the control unit 26 controls the units in the image processing apparatus 1 in accordance with the image processing program stored in the storage unit 10, thereby executing the processing shown in FIG. 3. This enables to register first time series volume data and second time series volume data for each phase, as in the embodiment. Note that in the modification, the first volume data is set to be wall motion volume data generated in real time upon echography. The second volume data is set to be 2D or 3D medical image data generated by an arbitrary medical image diagnostic apparatus. The medical image data is set to be, for example, volume data generated by the ultrasonic diagnostic apparatus 50, CT volume data generated by an X-ray CT apparatus, or X-ray contrast image data generated by an X-ray CT apparatus. These 2D or 3D medical image data are stored in the storage unit 10.

An example of the operation of the ultrasonic diagnostic apparatus 50 will briefly be described below. Note that the first volume data is assumed to be wall motion volume data, and the second volume data is assumed to be CT volume data.

The ROI setting unit 12 sets a wall motion ROI for time series wall motion volume data and a CT ROI, which is anatomically almost the same as the wall motion ROI, for time series CT volume data for each phase in accordance with a user instruction or by image processing. The associating unit 14 associates the wall motion ROI with the CT ROI for each phase. The registering unit 16 registers the time series wall motion volume data and the time series CT volume data for each phase based on the relative positional relationship between the wall motion ROI and the CT ROI which are associated with each other. The display image generation unit 18 generates time series wall motion display image data and time series CT display image data based on the registered time series wall motion volume data and time series CT volume data. The display unit 20 displays the wall motion display image data and the CT display image data in parallel or in a superimposed manner as moving images.

The above-described arrangement enables the ultrasonic diagnostic apparatus 50 of the modification to register time series image data generated in real time upon echography and another time series image data for each phase.

As described above, according to the modification, it is possible to provide an ultrasonic diagnostic apparatus and an image processing method capable of easily comparing identical portions included in different time series image data.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An image processing apparatus comprising:
a storage unit configured to store 2D or 3D first time series image data and second time series image data over a predetermined period;
a setting unit configured to set a first ROI on the first image data and a second ROI on the second image data for each of a plurality of phases in the predetermined period in accordance with a user instruction or by image processing, the second ROI being anatomically substantially the same as the first ROI;
an associating unit configured to associate the set first ROI with the set second ROI for each of the plurality of phases; and
a registering unit configured to register the first image data and the second image data for each of the plurality of phases based on a relative positional relationship between the first ROI and the second ROI which are associated with each other.

2. The apparatus according to claim 1, further comprising:
a generation unit configured to generate first display image data and second display image data based on the first image data and the second image data which are registered; and
a display unit configured to display the first display image data and the second display image data as dynamic image.

3. The apparatus according to claim 2, wherein the display unit displays the first display image data and the second display image data in parallel or in a superimposed manner.

4. The apparatus according to claim 1, wherein each of the first image data and the second image data is volume data.

5. The apparatus according to claim 1, wherein the first image data is volume data acquired by an ultrasonic diagnostic apparatus before stress echo, and the second image data is volume data acquired by the ultrasonic diagnostic apparatus after stress echo.

6. The apparatus according to claim 1, wherein one of the first image data and the second image data is volume data concerning a spatial distribution of wall motion information calculated by wall motion analysis.

7. The apparatus according to claim 1, wherein one of the first image data and the second image data is X-ray contrast image data generated by an X-ray diagnostic apparatus.

8. The apparatus according to claim 1, wherein at least one of the first display image data and the second display image data is at least one of an image generated by MPR processing and an image generated by volume rendering.

9. The apparatus according to claim 1, wherein the plurality of phases are all phases in the predetermined period.

10. The apparatus according to claim 1, wherein the setting unit sets the first ROI on the first image data and the second ROI on the second image data in accordance with a user instruction for at least one specific phase of the plurality of phases.

11. The apparatus according to claim 10, wherein the first ROI and the second ROI for the specific phase are set based on a plurality of feature points designated by a user in each of the first image data and the second image data.

12. The apparatus according to claim 10, wherein the setting unit sets, by image processing, the first ROI and the second region for a remaining phase other than the specific phase of the plurality of phases based on a position of the first ROI and a position of the second ROI in the specific phase.

13. The apparatus according to claim 12, wherein the associating unit associates the first ROI and the second region for the remaining phase based on the position of the first ROI and the position of the second ROI for the specific phase.

14. The apparatus according to claim 1, wherein the image processing is at least one of interpolation, extrapolation, automatic recognition, and tracking processing.

15. The apparatus according to claim 1, wherein each of the phases is set in accordance with a phase designated by a user on an electrocardiogram.

16. The apparatus according to claim 1, wherein if the first image data and the second image data have different time resolutions, the setting unit calculates one of a position of the first ROI and a position of the second ROI for an absence phase by one of interpolation and extrapolation.

17. The apparatus according to claim 2, wherein the display unit makes a size of the first ROI in the first display image data match a size of the second ROI in the second display image data based on the relative positional relationship between the first ROI in the first image data and the second ROI in the second image data.

18. The apparatus according to claim 2, wherein the display unit displays the first display image data and the second display image data as dynamic image while fixing a first display section of the first display image data and making a second display section of the second display image data follow the first display section.

19. The apparatus according to claim 2, wherein
the first display image data is functional image data generated by volume rendering from wall motion volume data generated by an ultrasonic diagnostic apparatus,
the second display image data is structural image data generated by volume rendering from CT volume data generated by an X-ray computed tomography apparatus, and
the display unit displays the functional image data and the structural image data while superimposing the functional image data on the structural image data.

20. The apparatus according to claim 19, wherein the display unit superimposes a wall motion abnormal region included in the functional image data on the structural image data.

21. The apparatus according to claim 20, wherein the display unit highlights a specific vascular region included in the structural image data and anatomically running through the wall motion abnormal region.

22. The apparatus according to claim 21, wherein the display unit displays the specific vascular region in one of a color, a lightness, and a saturation different from that of other vascular regions included in the structural image data.

23. The apparatus according to claim 21, wherein the display unit flashes the specific vascular region.

24. The apparatus according to claim 2, wherein
the first display image data is multisection functional image data generated by MPR processing from wall motion volume data generated by an ultrasonic diagnostic apparatus,
the second display image data is multisection structural image data generated by MPR processing from CT volume data generated by an X-ray computed tomography apparatus, and
the display unit displays the multisection functional image data and the multisection structural image data while superimposing the multisection functional image data on the multisection structural image data.

25. The apparatus according to claim 24, wherein a section position of the multisection functional image data and a section position of the multisection structural image data are set at each of an apex portion, an intermediate portion, and a base portion of a heart.

26. The apparatus according to claim 25, wherein the display unit displays superimposed image data of the functional image data and the structural image data for the apex portion of the heart, superimposed image data of the functional image data and the structural image data for the intermediate portion, and superimposed image data of the functional image data and the structural image data for the base portion of the heart in parallel as dynamic image.

27. The apparatus according to claim 26, wherein the display unit highlights a specific vascular region of vascular regions included in the structural image data.

28. The apparatus according to claim 26, wherein the display unit highlights a specific vascular region included in the structural image data and anatomically running through the wall motion abnormal region.

29. The apparatus according to claim 26, wherein the display unit highlights a peripheral region of a specific vascular region of vascular regions included in the structural image data.

30. The apparatus according to claim 2, wherein
the first display image data is functional image data generated by volume rendering from wall motion volume data generated by an ultrasonic diagnostic apparatus,
the second display image data is X-ray contrast image data generated by an X-ray diagnostic apparatus, and
the display unit displays superimposed image data of the functional image data and the X-ray contrast image data.

31. The apparatus according to claim 2, wherein
the functional image data includes first functional image data generated by volume-rendering the wall motion volume data at a viewpoint set outside a cardiac region and second functional image data generated by volume-rendering the wall motion volume data at a viewpoint set inside the cardiac region, and
the display unit displays first superimposed image data of the first functional image data and the X-ray contrast image data and second superimposed image data of the second functional image data and the X-ray contrast image data in parallel as dynamic image.

32. An image processing apparatus comprising:
a storage unit configured to store 3D first time series image data and 3D second time series image data concerning the same examination region;
a registering unit configured to register the first time series image data and the second time series image data for each phase based on a relative positional relationship between a first ROI included in the first time series image data and a second ROI included in the second time series image data, the second ROI being anatomically substantially the same as the first ROI; and
a display unit configured to display the registered first time series image data and the second time series image data in a superimposed manner or in parallel.

33. An ultrasonic diagnostic apparatus comprising:
an ultrasonic probe configured to transmit ultrasonic waves to an examination region of a subject, receive the ultrasonic waves reflected by the subject, and generate an echo signal corresponding to the received ultrasonic waves;
a generation unit configured to generate 2D or 3D time series echographic image data based on the echo signal;
a storage unit configured to store 2D or 3D time series medical image data concerning the examination region of the subject, the time series medical image data being generated by a medical image diagnostic apparatus;
a setting unit configured to set a first ROI on the time series echographic image data and a second ROI on the time series medical image data for each phase in accordance with a user instruction or by image processing, the second ROI being anatomically substantially the same as the first ROI,;
an associating unit configured to associate the set first ROI with the set second ROI for each phase; and
a registering unit configured to register the time series echographic image data and the time series medical image data for each phase based on a relative positional relationship between the first ROI and the second ROI which are associated with each other.

34. The apparatus according to claim 33, wherein the time series echographic image data concerns a spatial distribution of wall motion information calculated by wall motion analysis.

35. An image processing method comprising:
setting a first ROI on 2D or 3D first time series image data and a second ROI on 2D or 3D second time series image data for each phase in accordance with a user instruction or by image processing, the second ROI being anatomically substantially the same as the first ROI;
associating the set first ROI with the set second ROI for each phase; and
registering the first time series image data and the second time series image data for each phase based on a relative positional relationship between the first ROI and the second ROI which are associated with each other.

36. An image processing method comprising:
registering 3D first time series image data and 3D second time series image data for each phase based on a relative positional relationship between a first ROI included in the first time series image data and a second ROI included in the second time series image data, the second ROI being anatomically substantially the same as the first ROI; and
displaying the registered first time series image data and the second time series image data in a superimposed manner or in parallel.
